(19) United States
(12) Patent Application Publication
(10) Pub. No.: US 2004/0267137 A1
(43) Pub. Date: Dec. 30, 2004

(54) Apparatus and method for IC-based ultrasound transducer temperature sensing

(76) Inventors: Michael Peszynski, Newburyport, MA (US); Bemard Savord, Andover, MA (US)

Correspondence Address: PHILIPS INTELLECTUAL PROPERTY & STANDARDS, P.O. BOX 3001, BRIARCLIFF MANOR, NY, 10510, US

(21) Appl. No.: 10/878,144

(22) Filed: Jun. 28, 2004

Related U.S. Application Data
(60) Provisional application No. 60/482,948, filed on Jun. 27, 2003.

(51) Int. Cl. A61B 8/00

(52) U.S. Cl. 600/459

(57) ABSTRACT

An apparatus and method are provided for temperature sensing in ultrasound imaging devices. The ultrasound imaging apparatus includes at least one temperature sensitive device positioned in thermal communication with the heat producing regions of the ultrasound imaging device to sense the operating temperature of at least one ultrasound transducer. The temperature data is used by monitoring and control systems to warn a user or to regulate the temperature automatically.

### CROSS REFERENCE TO RELATED CASES

Applicants claim the benefit of Provisional Application Ser. No. 60/482,948, filed Jun. 27, 2003.

### FIELD OF THE INVENTION

The present invention relates generally to medical devices. More particularly, the present invention relates to an apparatus and method for monitoring and controlling heat produced by ultrasound medical devices.

### DESCRIPTION OF THE RELATED ART

Medical ultrasound imaging has become a popular means for visualizing and medically diagnosing the condition and health of interior regions of the human body. With this technique, an acoustic transducer probe, which is attached to an ultrasound system console via an interconnection cable, is held against the patient's tissue by the sonographer where it emits and receives focused ultrasound waves in a scanning fashion. The scanned ultrasound waves, or ultrasound beams, allow the systematic creation of image slices of the patient's internal tissues for display on the ultrasound console. The technique is generally quick, painless, fairly inexpensive and safe, even for such uses as fetal imaging.

In order to get the best performance from an ultrasound system and its associated transducers, it is desirable that the transducers used to emit and receive ultrasonic pulses be capable of operating at the maximum acoustic intensity allowable by the U.S. Food and Drug Administration (FDA). This will help maximize the signal to noise ratio for the given system and transducer, help achieve the best possible acoustic penetration, and ensure that imaging performance is not limited by the inability to emit the full allowable acoustic intensity. At the same time, there are practical and regulatory limits on the allowable surface temperature that the transducer may attain as it performs its imaging functions. The Underwriters Laboratory (U.L.) Standard #UL544 “Standard for Safety: Medical and Dental Equipment” specifies an upper limit of 41° C. for the transducer portion contacting the patient's skin. In addition, sonographers prefer to grip a transducer case which is comfortably cool, thereby minimizing perspiration on their hands and a potential to lose their grip on the device.

Given that it is desirable to be able to operate at the maximum allowable acoustic intensity and also desirable to control the surface temperature distribution of the patient and user-contacting portions of the transducer's surfaces, thermal engineering is a serious consideration during transducer design. There are essentially two possible paths to proceed with regard to transducer thermal engineering.

The first path makes use of passive cooling mechanisms and involves insuring that the heat that is generated both by the electro-acoustic energy conversion process taking place in the transducer's piezoelectric elements and by the acoustic energy passing through and/or into adjacent transducer materials is passively spread out to as large an external transducer surface area as possible. This heat spreading process is typically achieved internal to the transducer by thermal conduction through solid materials and subsequently from the transducer's external case employing natural free convection to the atmosphere. Ideally, the external heat conducting surface area would consist of the entire transducer's external surface area from which free convection cooling to the atmosphere can potentially take place in an unobstructed manner. Transducer manufacturers have thus incorporated various passively conducting heat-spreading plates and members inside the transducer's interior spaces to ensure the spreading of the heat to the entire transducer case surface. However, it is the ability to get the heat out of the electro-acoustic elements themselves and into those adjacent internal thermal-sinking structures that provides a significant portion of the probes total thermal dissipation resistance. If this internal thermal path is not a good one it is difficult to spread the heat generated by the piezoelectric elements around the case. If the heat generated by the piezoelectric elements cannot be removed, and effectively coupled and sunk to the entire transducer case area, then the probe surface portion in contact with the patient runs hotter than desired as this probe portion is directly adjacent the piezoelectric elements. Thus, even in the passive strategy, there is a concern regarding three key mechanisms: a) removing the heat from the highly localized piezoelectric elements region; b) spreading said heat efficiently to the external case surfaces; and c) allowing for unobstructed natural convection from the warm transducer surfaces.

In any event, using this passive strategy, maximizing the external probe surface area onto which heat spreads in a fairly uniform manner minimizes the peak surface temperature attained anywhere on the probe's surface during steady state convection of the probe's heat to the ambient environment. This passive strategy amounts to spreading the heat load around to minimize the impact of the limited ability of free convection to dissipate heat. Its fundamental limitation is that, for most transducers, even if heat is spread uniformly on the external case surfaces, it only takes a few watts of transducer driving power to cause the average transducer surface temperature to become unacceptable either with respect to the patient or the sonographer. In these cases, and particularly for small transducers having small surface areas, one may find that one is unable to operate at the allowable acoustic intensity limit because of excessive temperatures. Additionally, the patient's body temperature also affects the overall temperature of the transducer, especially in the case of febrile patients.

An extension of the passive-cooling approach has included an attempt to conduct or spread some of the heat down the length of the attached cable in order to permit the cable to offer more passive convection surface area. This helps the situation only incrementally because of the user-preferred small diameter cable and the difficulty of providing much of a thermally conductive path in such a small diameter cable without compromising the desired flexibility and compactness of the cable. Such an incremental measure is described in U.S. Pat. No. 5,213,103 “Apparatus for and method of cooling ultrasonic medical transducers by conductive heat transfer” to Martin, et al.

It should be noted that for endoscopic transducers (probes inserted internally into the human body), heat is dissipated both by direct conduction to the patient's internal tissues and fluids, as well as by conduction through the cable and convection from the exposed transducer handle which remains external to the patient's cavity. One must also control the maximum surface temperatures attainable by these probes.

The second strategy for cooling transducers, described in U.S. Pat. No. 5,560,362 is to utilize active cooling rather than passive cooling, in order to dissipate heat well beyond that which can be passively conducted from the external transducer surfaces. Active cooling implies that someone provides a means to actively remove heat from the transducer, such as by employing a pumped coolant or other active refrigeration means. Using active cooling ensures that a user is always able to operate the acoustic transducer up to the allowable acoustic intensity limit while also maintaining acceptable surface temperatures regardless of how small the transducer is or how much surface area it offers for cooling relative to its acoustic intensity.

It is worth noting, several other techniques are also employed to control the transducer temperature in which an overt action is performed, such as adjusting ultrasound intensity and modulating duty cycle in response to temperature changes. Within the scope of the present invention theses methods of temperature control are considered active cooling methods also.

Regardless of the cooling method employed, either passive or active, reliable temperature monitoring of the ultrasound radiating surface and other surfaces in contact with the patient is required whether to simply notify the sonographer of temperature conditions or to actually implement active cooling procedures. Temperature sensing devices are routinely employed in medical ultrasound imaging devices, however, these sensors, usually thermistors or thermocouples, have limitations. These sensors are bulky in comparison to sizes routinely obtained in integrated circuit fabrication, where dimensions on the order of a few millimeters containing numerous electronic elements are easily obtainable. This bulkiness impacts their placement as their size can interfere with the proper transmission of ultrasonic energy to the patient. Their size and shape also preclude them from being placed at the point of maximum temperature rise—usually at the center of the acoustic window. Additionally, the wiring and interconnects required to effectively utilize thermistors or thermocouples have a potential for non-reliability and in life critical situations, such as Trans-Esophageal Echocardiography imaging during cardiac surgery, such a sensor failure resulting in system shutdown due to over-heating can have serious consequences.

### SUMMARY OF THE INVENTION

The present invention provides an array of temperature sensing circuits that are constructed integrally to an Integrated Circuit (IC) used to sense the patient-transducer interface temperature. The IC is in thermal communication with heat-generating surfaces. The multi-circuit nature of the inventive surface temperature monitoring method allows for the gathering of data on the distribution of temperature across the entire heat-generating surface rather than simply at one discrete point.

The present invention is applicable in both passive and active cooling systems to provide feedback to the cooling mechanisms as exemplified in U.S. Pat. No. 6,709,392, assigned to a common assignee as the present invention and herein incorporated by reference in its entirety. The temperature data provided by the present invention can be used to activate a temperature warning indicator in a passive or active system. The present invention can also control the active cooling methods employed—activating the cooling mechanisms when a certain temperature threshold is surpassed and shutting off the mechanism when the transducer is operating at acceptable temperatures. Additionally, the temperature sensing circuit of the present invention can be used within a circuit designed to perform various other temperature control techniques, i.e. changing the transducer duty cycle, adjusting output power of the transducer or shutting down the ultrasound transducer, allowing the transducer to cool down and avoiding any injury to the patient due to exposure to high temperatures.

The ultrasound imaging apparatus of the present invention houses one or more ultrasound transducers appropriately configured to emit ultrasonic energy to the target area to be imaged. Additionally, a temperature sensing array consisting of one or more semiconductor-based temperature sensors, fabricated as part of an IC, are situated in a manner so as to be in thermal communication with areas within the ultrasound imaging apparatus that produce heat, specifically the ultrasound transducer's energy radiating surface. The temperature information gathered—continuously or at predetermined intervals—is subsequently relayed to temperature display and control circuitry and mechanisms. If temperature fluctuations cause the monitored areas to operate at a temperature outside a predetermined operating temperature range, the control circuitry and mechanisms perform at least one action which will result in the out of range region to return to a temperature within the predetermined operating temperature range.

### BRIEF DESCRIPTION OF THE DRAWINGS

For a better understanding of the invention, reference is made to the following description of preferred embodiments thereof, and to the accompanying drawings, wherein:

FIG. 1 is an exemplary prior art ultrasound imaging probe;

FIG. 2 is an enlarged phantom view of the prior art imaging probe tip shown by FIG. 1;

FIG. 3 is an ultrasound imaging probe in accordance with the present invention;

FIG. 4 is an enlarged phantom view of the imaging probe tip shown by FIG. 3;

FIG. 5 is a detailed view of the ultrasound transducer and temperature sensing apparatus in accordance with the present invention; and

FIG. 6 is a typical derating response characteristic of a standard silicon diode in accordance with the present invention.

### DETAILED DESCRIPTION OF THE PREFERRED EMBODIMENTS

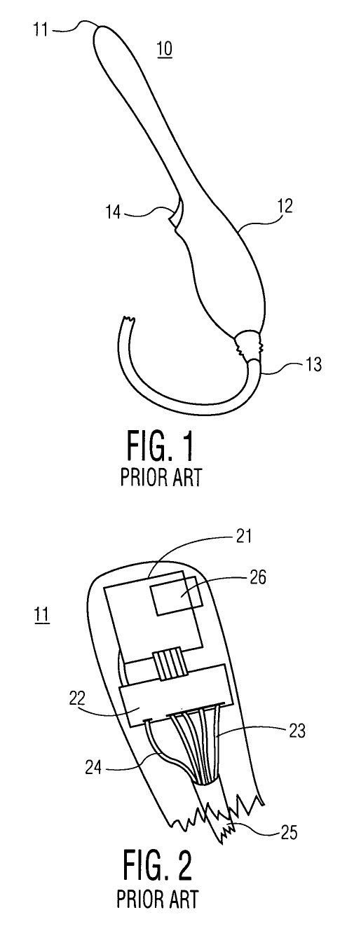

Referring to FIGS. 1 and 2, currently available medical ultrasound probes 10 (see FIG. 1) generally consist of a distal tip 11 which houses an ultrasound transducer 21 and associated control circuitry 22. Additionally, a temperature sensor or cooling apparatus 26 is present and in thermal contact with the heat producing elements, such as the ultrasound transducer 21. As discussed previously, currently available medical ultrasound probes need to operate within strict temperature limits because of the risk of tissue damage to the patient being imaged. The components housed in the distal tip 11 are connected to the ultrasound system (not shown) through a cable 25 containing control conductors 23 for controlling the ultrasound transducer 21, temperature sensor/cooling apparatus 26 and control circuitry 22 and power conductors 24 for supplying power to the various components. The handle 12 segment of the ultrasound probe 10 may house an activator 14 to activate the ultrasound transducer etc. and a cable 13 connects the ultrasound probe 10 to the ultrasound system (not shown). The activator may be of the form of a trigger, button or other appropriate means.

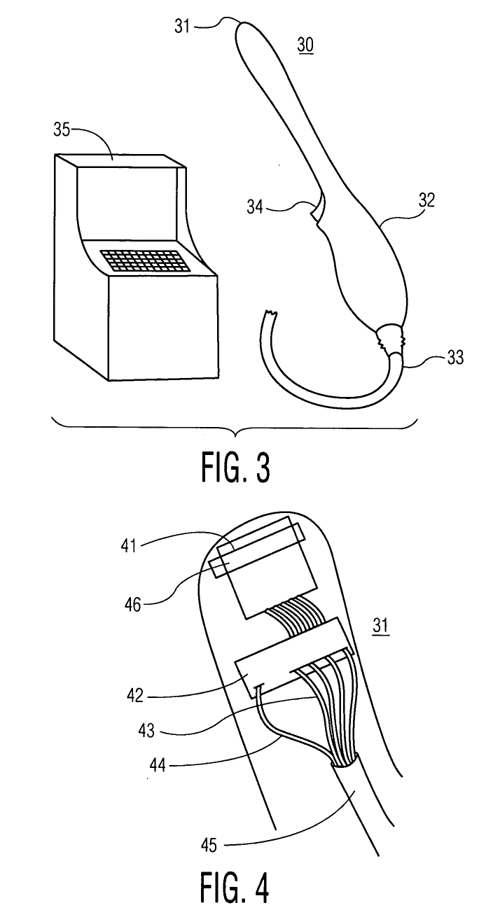

The preferred embodiment, as illustrated in FIG. 3, includes many of the same elements as the prior art ultrasound probe 10 (see FIG. 1). The distal tip 31 is connected to a handle segment 32 of the probe 30. An activator 34 for activating the ultrasound transducer 41 (see FIG. 4) is located on the handle 32 and a cable for connecting the probe to an ultrasound system 35. As before, the activator may take the form of a trigger, button or other such appropriate device. The ultrasound system 35 should preferably include a display console with a device for inputting commands, i.e. keyboard, pointing device, etc., connector for supplying control signals to and receiving image data from the probe 30.

As illustrated in FIG. 4, the probe's distal tip 31 houses an ultrasound transducer 41, preferably in thermal communication with the inventive temperature sensing assembly 46 and additional support circuitry 42. These components are connected to and controlled by an ultrasound system 35 (see FIG. 3) through a cable 45 consisting of a plurality of wires supplying power and control signals to the various components and transmitting the received transducer signals to the ultrasound system 35.

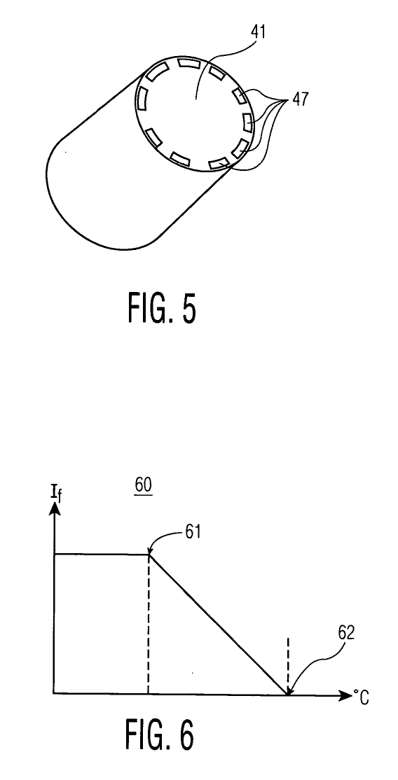

FIG. 5 illustrates an enlarged and more detailed view of the ultrasound transducer 41 and temperature sensing assembly 46 (see FIG. 4). Preferably, the temperature sensing assembly 46 includes at least one IC 47, containing one or more temperature sensitive devices arranged in an array to provide feedback from various points on the heat-producing surface. The temperature sensitive device may be any of the following semiconductor devices: diodes, resistors, thermistors, transistors or other parts having a substantially linear voltage-temperature relationship, however the diode is preferably used in the preferred embodiment. Additionally, non-linear and threshold voltage-temperature relationships may be used in the present invention.

The IC placement depicted in FIG. 5 is for illustrative purposes only and should not be construed to imply a limitation to the only array configuration shown; any of a host of array configurations, i.e. two bisecting lines of sensors, pseudo-random, and grid, etc., should be considered as part of the present embodiment. The diode's sensitivity to changes in ambient temperature and IC's compact size make it ideal for use as a temperature sensor within the tight confines of an ultrasound probe. The compact form factor possible with an IC allows placement of the sensor closer to hotspots on the transducer while still allowing the ultrasound waves to propagate to the patient with minimal interference than is possible with the bulkier thermistors or thermocouples currently being used. This close proximity to the source of maximum temperature rise allows the IC-based temperature sensor to provide a more accurate indicator of ultrasound transducer performance and compliance with safe operating parameters.

A diode's temperature response also know as the Current Derating Curve 60 (see FIG. 6) indicates the relation of the diode's forward current as a function of ambient temperature. As shown in FIG. 6, an increase in the ambient temperature around the diode 47 causes a decrease in the current flow through the diode 47. This temperature/current relation is essentially linear, which makes it a very good indicator of temperature. Additionally, diodes are designed with a wide range of derating curves 60 so many models exist that meet nearly any temperature sensing need and can be customized during the IC fabrication process. In the case of the present embodiment, however, the diode IC 47 used should preferably have a derating curve 60 that begins 61 at or below 35° C. and cuts off 62 at 42° C. or above. These values are specifically selected to cover the appropriate operating range of the device as bounded by a patient's normal body temperature of 37° C. and the UL Standard #UL544 upper limit of 41° C. as safe for medical and dental equipment. However, in certain cases the ultrasound device may be utilized in an environment in which the patient's temperature may be significantly lowered i.e. during certain types of medical procedures in which lowered body temperature is desirable; in such a case the derating curve 60 cutoff points 61 and 62 may differ from those indicated above.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law

1. An ultrasonic imaging apparatus comprising:
at least one ultrasound transducer, wherein said ultrasound transducer is positioned such that emitted ultrasonic energy is transmitted through an acoustic window and;
at least one temperature sensitive device disposed within an IC positioned in thermal communication with heat producing regions of said at least one ultrasound transducer, wherein the temperature sensitive device contains at least one semiconductor device having a voltage-temperature relationship; and
an assembly for monitoring and controlling the operating temperature of said at least one ultrasound transducer in accordance with the temperature sensed by said at least one temperature sensitive device such that said temperature is within a predetermined operating temperature range.

2. An ultrasonic imaging apparatus as in claim 1, wherein said voltage-temperature relationship is substantially linear.

3. An ultrasonic imaging apparatus as in claim 1, wherein said at least one semiconductor device is selected from the group including: diode, thermistor, resistor and transistor.

4. An ultrasonic imaging apparatus as in claim 1, wherein said temperature sensitive device is located in proximity to heat generating regions of the at least one ultrasound transducer and/or the acoustic window and arranged in an array configuration such that said at least one temperature sensitive device monitors temperature fluctuations of said regions and/or said acoustic window.

5. An ultrasound imaging apparatus as in claim 1, wherein said at least one temperature sensitive device is arranged and affixed in a predetermined location.

6. An ultrasound imaging apparatus as in claim 1, wherein said predetermined operating temperature range is about 35° C. to about 41° C.

7. An ultrasound imaging apparatus as in claim 1, wherein said assembly controls the operating temperature by performing at least one action selected from the group consisting of indicating a warning to a user, initiating an active cooling mechanism, and at least partially reducing power to the at least one ultrasound transducer.

8. A temperature sensing and control system for an ultrasound imaging apparatus comprising:
at least one temperature sensitive device disposed within an IC positioned in thermal communication with heat producing regions of an at least one ultrasound transducer, wherein the temperature sensitive device contains at least one semiconductor device having a voltage-temperature relationship;
means for receiving temperature related data from the at least one temperature sensitive device; and
means for utilizing said received temperature related data and maintaining the at least one ultrasound transducer within a predetermined operating temperature range.

9. A temperature sensing and control system as in Clam 8, wherein said voltage-temperature relationship is substantially linear.

10. A temperature sensing and control system as in Clam 8, wherein said at least one temperature sensitive device is located in proximity to heat generating regions of the at least one ultrasound transducer and arranged in an array configuration such that said at least one temperature sensitive device monitors temperature fluctuations of said regions.

11. A temperature sensing and control system as in Clam 8, wherein said at least one semiconductor device is selected from the group comprising of: diode, resistor, thermistor and transistor.

12. A temperature sensing and control system as in Clam 8, wherein said predetermined operating temperature range is about 35° C. to about 41° C.

13. A temperature sensing and control system as in claim 8, wherein said means for utilizing said received temperature related data and maintaining the at least one ultrasound transducer within a predetermined operating temperature range includes means for performing at least one action selected from the group including: sending a warning to a user, initiating an active cooling mechanism, and at least partially reducing power to the at least one ultrasound transducer.

14. A method for sensing and controlling an operating temperature of an ultrasound imaging apparatus having at least one ultrasound transducer, said method comprising the steps of:
providing at least one temperature sensitive device disposed within an IC positioned in thermal communication with heat producing regions of an at least one ultrasound transducer, wherein the temperature sensitive device contains at least one semiconductor device having a voltage-temperature relationship;
sensing temperature of said at least one ultrasound transducer using said at least one temperature sensitive device; and
determining whether to initiate at least one temperature control action for maintaining the temperature of the at least one ultrasound transducer within a predetermined operating temperature range, wherein said determination is based on said sensed temperature.

15. A temperature sensing and control system as in Clam 14, wherein said voltage-temperature relationship is substantially linear.

16. A method for sensing and controlling an operating temperature as in Clam 14, wherein said at least one semiconductor device is selected from the group comprising of: diode, resistor, thermistor and transistor.

17. A method for sensing and controlling an operating temperature as in Clam 14, wherein said at least one temperature sensitive device is disposed within an IC and arranged in an array configuration.

18. A method for sensing and controlling an operating temperature as in Clam 14, wherein said predetermined operating temperature range is about 35° C. to about 41° C.

19. A method for sensing and controlling an operating temperature as in claim 14, wherein said at least one temperature control action is selected from the group including: sending a warning to user, initiating an active cooling mechanism, and at least partially reducing power to the at least one ultrasound transducer.

20. A method for sensing temperature and controlling an operating temperature as in claim 14, further comprising the step of initiating the at least one temperature control action if the sensed temperature is outside said predetermined operating temperature range.
